(19) United States
(12) Patent Application Publication
(10) Pub. No.: US 2014/0081193 A1
(43) Pub. Date: Mar. 20, 2014

(54) METHOD AND SYSTEM FOR ADJUSTING ROTATION SPEED OF AN EXTRACORPOREAL THERAPEUTIC DEVICE

(71) Applicants: Thomas I. Watters, Rochester, NY (US); Victor I. Ibarluzea, Brockport, NY (US); Daniel J. Di Girolamo, Miami, FL (US)

(72) Inventors: Thomas I. Watters, Rochester, NY (US); Victor I. Ibarluzea, Brockport, NY (US); Daniel J. Di Girolamo, Miami, FL (US)

(73) Assignee: Ortho-Clinical Diagnostics, Inc., Rochester, NY (US)

(21) Appl. No.: 13/615,777

(22) Filed: Sep. 14, 2012

(51) Int. Cl. A61M 1/36

(52) U.S. Cl. CPC A61M 1/3663
USPC 604/4.01

(57) ABSTRACT

An extracorporeal therapeutic device automatically controls a rotation speed of a centrifuge for processing whole blood. Separation of whole blood components is achieved with minimal thermal contribution to blood temperature from the motor control mechanism that rotates the centrifuge and from aerodynamic drag on the rotating centrifuge.

### FIELD OF THE INVENTION

The invention relates generally to adjusting a rotation speed of an extracorporeal therapeutic device and, in particular, to adjusting the rotation speed of a centrifuge in an extracorporeal therapeutic device in response to a blood collection rate.

### BACKGROUND OF THE INVENTION

Extracorporeal photopheresis is a therapeutic procedure performed outside the body of a patient using an extracorporeal therapeutic device to withdraw a volume of whole blood that is then centrifuged to separate the white blood cells from the red blood cells and plasma. The red blood cells and plasma are immediately returned to the patient. The white blood cells are treated with methoxsalen which is photoactivated with exposure to ultraviolet-A light. The treated white blood cells are then reinfused into the patient. The combination of methoxsalen and ultraviolet-A light causes the white blood cells to undergo apoptosis. Reinfusion of these white blood cells into the patient can help modulate the patient's immune system. This entire photopheresis process occurs in a closed blood circuit. This reduces the potential for infections and ensures that patients have their autologous cells returned to them.

An extracorporeal therapeutic device is able to operate in either a single-needle or double-needle mode. The double-needle mode significantly reduces extracorporeal photopheresis time because it enables continuous blood collection and return from and to the patient, i.e., a continuous flow mode. However, if venous access is compromised or difficult to obtain, then the operator can also use the extracorporeal therapeutic device in a discontinuous flow/single-needle mode, where blood collection and return are accomplished via the same venous access site. Therefore, operation of the device can easily be changed from double-needle to single-needle mode.

An extracorporeal therapeutic device automatically isolates the leukocyte fraction in both flow modes (continuous or discontinuous). During continuous-flow separation, the extracorporeal therapeutic device maintains the expanding leukocyte fraction in the centrifuge, enabling single harvest collection. The constant spinning of the centrifuge allows separation, which results in highly efficient leukocyte collection and reduced treatment times.

At present, the use of extracorporeal photopheresis is made in treating a number of diseases including the following:

Graft-versus-host disease occurs in allogeneic bone marrow transplants, which involve transplanted cells from a donor other than the patient. It is a life-threatening complication in which the new donor cells attack the patient's organs and tissues. The result is an inflammatory reaction targeted against the skin, mouth, lungs and liver that results in severe tight skin, mouth ulcers, difficulty breathing, liver failure, and in severe cases, death. Acute Graft-versus-host disease starts within the first 3 months after transplant. Chronic Graft-versus-host disease starts more than three months after transplant, and can last for as long as three years. According to the International Center for Blood and Marrow Transplant Research, of the 14,000 allogeneic transplants performed worldwide, approximately 50 percent of patients will develop acute Graft-versus-host disease, and another 50 percent to 70 percent will develop chronic Graft-versus-host disease.

Cutaneous T-cell lymphoma is a slowly progressive form of cancer that has been increasing in incidence. Patients may experience symptoms of thickened, red, cracking, scaling or intensely itchy skin in localized areas or all over the body. About 10 percent of patients will have blood, lymph node and/or internal organ involvement with serious complications. Many patients live normal lives during treatment and some are able to remain in remission for long periods of time. There is a greater frequency among men than women and Cutaneous T-cell lymphoma is more common after the age of 50.

In general, the steps involved in photopheresis processing are outlined as follows:

Step 1: While removing whole blood from the patient, the device centrifuges the blood to separate red blood cell from the leukocyte-containing buffy coat cells.

Step 2: The buffy coat cells remain in a photopheresis device, such as, for example, the THERAKOS™ Photopheresis System, while other components are returned to the body.

Step 3: The buffy coat cells are treated with methoxsalen.

Step 4: The buffy coat cells are exposed to an automatically calculated amount (approximately 1.5 to 2 Joules/cm2) of ultraviolet-A light to activate the methoxsalen.

Step 5: The treated cells are then reinfused into the patient.

Certain known commercial extracorporeal therapeutic devices, such as THERAKOS™ brand photopheresis systems, have an interactive and informative operator interface. This interface enables the operator to respond immediately to the patient's needs. The interface, while fully automated, also has a manual override. The ability to manually override the photopheresis process allows for customized blood collection and, during buffy coat collection, the operator may expand the real-time hematocrit chart to monitor automatic hematocrit detection or gain assistance with manual intervention. Such interfaces display real-time extracorporeal photopheresis data in graphics and text; including processed blood volume, collect and return rates with corresponding line pressures, and fluid balance limits. The operator is able to direct all the treatment phases by observing and adjusting a plurality of parameter settings via a touch screen keypad. The operator is assisted by on-screen messages, which immediately display recommended corrective actions during warning alarm and error alarm notifications.

Further details of extracorporeal therapeutic devices and the associated photopheresis processing are discussed in Knobler, et al., Extracorporeal Photopheresis: Past, Present, and Future, Journal American Academy of Dermatology 2009, Vol. 61, pp. 652-665 (Aug. 10, 2009); also in Oliven et al., Extracorporeal Photopheresis: A Review, Blood Reviews, Vol. 15, pp. 103-108 (2001); and also in Foss et al., Mini-review: Extracorporeal Photopheresis in Chronic Graft-Host Disease, Bone Marrow Transplantation, Vol. 29, pp. 719-725 (2002), each of which are herein incorporated by reference in their entireties.

Additional details of extracorporeal therapeutic devices and the associated photopheresis processing can also be found in Briggs et al., U.S. Pat. No. 7,914,477, March 2011; also in Briggs, Dennis, U.S. Pat. No. 7,850,634, December 2010; and also in Hutchinson et al., U.S. Pat. No. 7,465,285, December 2008, each of which are herein incorporated by reference in their entireties.

Of particular importance during the photopheresis process is the ability to monitor and contain within limits the temperature of the whole blood contained in the inner container of the therapeutic system. This inner container is usually the centrifuge, which as previously discussed, has the function of separating the leukocyte fraction, i.e. the white blood cell fraction, from the whole blood for further processing. However, the aerodynamic drag of the centrifuge along with the mechanical friction of the drive motor makes the centrifuge a heat source, which tends to raise the temperature of the blood during processing.

It is important that the temperature of the whole blood being processed is not allowed to increase to a point in which hemolysis could occur. For example, during patient treatment, a centrifuge bowl is spun at approximately 4800 rpm to produce centripetal forces of about 1500 g. This speed will allow separation of blood components to occur at a maximum blood collection rate of 100 ml/min. However, the high centrifuge speed increases temperature in the system due to mechanical friction and aerodynamic drag. Thus, if the whole blood collect rate is lowered, the treatment time is extended and temperatures within the system may rise to the point where damage to the blood cells could occur. Apparatuses and methods for adequately controlling heat sources that contribute to raising the temperature of the blood have not been fully explored. Devices have been developed that require various sensors to be attached which measure temperatures of the device, but these improvements do not control the heat that is generated. Rather, they are designed as monitoring devices that issue status indications and warnings.

### SUMMARY OF THE INVENTION

This application discloses an apparatus and method to automatically control a rotation speed of the centrifuge in an extracorporeal therapeutic device for minimizing its thermal effects on blood being processed. Empirically, a relationship between centrifuge speed reduction and a percentage yield of white blood cells from processed whole blood has been established. This relationship in combination with monitoring an operator selected collection rate of the blood provides a predetermined optimal rotation speed of the centrifuge that is effective for separating the white blood cell fraction, while avoiding undesired heat generation. The optimal rotation speed corresponds to the collection rate and the system automatically sets the speed of rotation of the centrifuge at the optimal rotation speed.

One aspect of the present disclosure is directed to a method of monitoring operation of an extracorporeal therapeutic device for processing whole blood. The method includes automatically determining a collection rate of whole blood in the extracorporeal therapeutic device and automatically determining a rotation speed of a centrifuge in the device corresponding to the collection rate. The rotation speed is then automatically set at the rotation speed corresponding to the collection rate.

Another aspect of the invention comprises an extracorporeal therapeutic system for processing whole blood. The system comprises an operator interface for an operator to input a selected collection rate, an intake for receiving the whole blood at the operator selected collection rate, a rotating centrifuge for separating components of the whole blood that is collected, a motor mechanically connected to the centrifuge and automatically controlled to cause the motor to rotate the centrifuge at a predetermined rotation speed corresponding to the operator selected collection rate.

Further objects, features, and advantages of the present application will be apparent to those skilled in the art from detailed consideration of the embodiments that follow.

### BRIEF DESCRIPTION OF THE DRAWINGS

FIG. 1 is a schematic diagram of an extracorporeal therapeutic device having a variable speed control system incorporated therein.

FIG. 2 is a graph of a blood collection rate and a rotation speed of the centrifuge.

FIG. 3 is a table of blood collection rates corresponding to centrifuge rotation speeds according to the linear relationship in FIG. 2.

FIG. 4 is flowchart of the extracorporeal therapeutic system monitoring logic.

### DETAILED DESCRIPTION OF THE PREFERRED EMBODIMENT

While the present invention is described with respect to the preferred embodiments described below and shown in the figures, the present invention is limited only by the metes and bounds of the claims that follow.

The apparatus and methods described herein enable effective control over preventable heating of the whole blood within an extracorporeal therapeutic device and enable the operator to select a collection rate for the associated photopheresis process that is most preferable for the patient.

For a general understanding of the disclosed technology, reference is made to the drawings. In the drawings, like reference numerals have been used to designate identical elements. In describing the disclosed technology, the following term(s) have been used in the description.

The term “collection rate” refers to a rate that blood is drawn from a patient undergoing photopheresis, measured in units of milliliters per minute (ml/min). Typically, the collection rate is set by an operator of the system anywhere from 5 ml/min to 100 ml/min in increments of 5 ml/min.

The term “rotation speed” refers to a rotation speed of the centrifuge bowl in the extracorporeal therapeutic device.

One aspect of this disclosure is directed to an apparatus and method for real-time monitoring of the collection rate of whole blood undergoing photopheresis processing in an extracorporeal therapeutic device and adjusting a centrifuge rotation speed in response thereto.

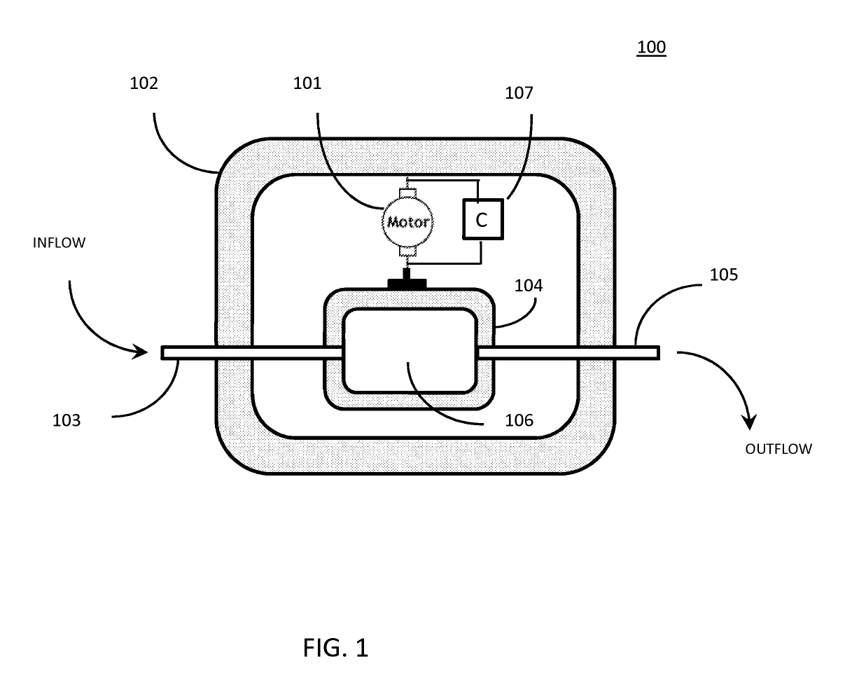

FIG. 1 is a schematic diagram of an extracorporeal therapeutic device 100 showing each of the major subcomponents of the device in a preferred embodiment. The device 100 comprise an outer casing wall 102 and inner container 104, which is usually a centrifuge. Whole blood is processed in the centrifuge 104 with internal volume 106. Unprocessed whole blood enters via the first connection (inflow) 103, at a collection rate selected by the operator, and processed whole blood exits (outflow) via a second connection 105 at a rate that is dependent on the selected collection rate. A motor 101 controls a rotation speed of the of the centrifuge and is in electrical communication with a motor controller 107 that controls the speed of the motor. Motor controller 107 includes a processor and standard on board memory for storing, executing, and processing control programs and data that are accessed by the motor controller 107 as programmed to automatically adjust a speed of the motor.

The thermal capacity of the air inside the outer casing wall 102 has been determined to be negligible, but it does allow heat transfer between the centrifuge 104 and the outer casing wall 102 by natural convection. Also, the extracorporeal therapeutic device loses heat from the outer casing wall 102 to the surrounding air by natural convection when the temperature of the outer casing wall 102 is greater than the temperature of the surrounding air. The primary source of thermal energy: input is associated with the operation of the centrifuge 104, which is a centrifuge rotating in mechanical connection to the electric motor 101. As the motor 101 increases in speed, the centrifuge 104 rotation speed increases. The thermal energy given off by the mechanical operation of the motor and the centrifuge increases with their rotational speed.

In addition to the thermal energy given off by the operation of the motor 101, there is air friction associated with the spinning of the centrifuge bowl 104 that produces additional thermal energy. This thermal input also increases with higher rotational speed of the centrifuge 104. This input thermal energy is subsequently transferred via conduction to the whole blood being processed by the centrifuge 104 and to the air contained within the outer casing wall 102. This transfer of thermal energy to the patient's whole blood results in a rise in temperature of the whole blood. As a result, if the whole blood temperature is able to increase to substantially about 45° Celsius, the possibility of hemolysis is greatly increased. Hemolysis is the rupturing of erythrocytes (red blood cells) and the release of their contents (hemoglobin) into the surrounding fluid, the blood plasma. Hemolysis therefore destroys the red blood cells and their oxygen-carrying ability, potentially resulting in anemia or more serious health-related conditions once the blood plasma and red blood cells are re-introduced to the patient. Hence, it is important to control the thermal input of the centrifuge's mechanical operation during the photopheresis process to insure that undesired increases in temperature of the patient's whole blood is prevented.

As explained above, during patient treatment the centrifuge 104 is spun at a predetermined rate during whole blood collection rates and allows separation of blood components to occur. If the whole blood collect rate is lowered, the treatment time is extended and the temperature of the blood within the system may rise to the point where damage to the blood cells could occur. An empirically derived relationship between the whole blood collection rate and the rotation speed of the centrifuge is used in the motor controller 107 to help prevent possible blood overheating and hemolysis of the patent's whole blood during photopheresis processing. As described above, as the collection rate (inflow) decreases in the extracorporeal therapeutic device, so does the processed blood outflow. Hence, the amount of time that the blood is retained in the centrifuge 104 increases. In this situation, where the blood collection rate is decreased, it is advantageous to lower the rotation speed of the centrifuge in order to decrease the amount of heat it produces.

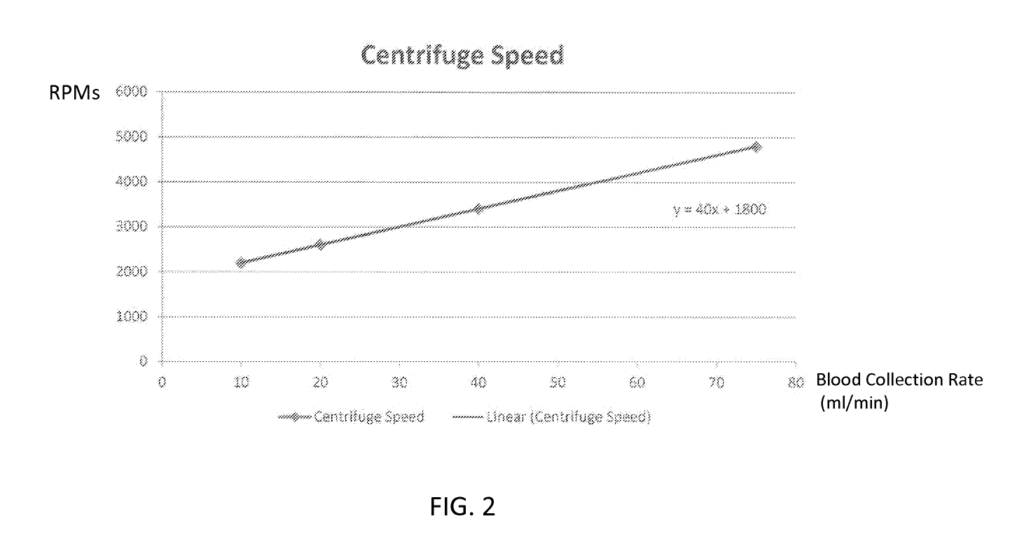

FIG. 2 depicts a graph of centrifuge rotation speed, y-axis, in revolutions per minute (RPM) vs. whole blood collection rates, x-axis, in milliliters per minute (ml/min). This empirical data was obtained from testing centrifuge rotation speeds at different collection rates. White blood cell yields were measured, with no white blood cells lost, and were found comparable to previous yields obtained using photopheresis devices in which no rotation speed adjustments were made. These data points demonstrate that reduction in centrifuge rotation speeds for collection rates below maximum collection rates are feasible for photophoresis processing. The empirical data obtained herein shows a linear relationship between blood collection rate (x-axis) and a rotation speed of the centrifuge (y-axis). The derived linear equation is y=40x+1800. Generally, for a corresponding collection rate, rotational speeds above the line shown in FIG. 2 can lead to a possibly increased hemolysis risk, however, a lower ambient temperature can have a decreasing effect on this risk. Rotational speeds below the line can lead to less than optimal separation times.

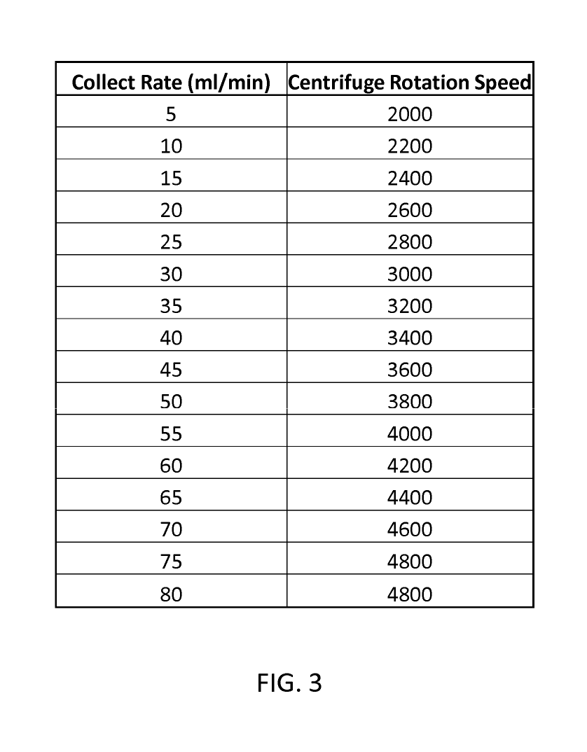

FIG. 3 presents a tabular listing represented by the above linear relationship (FIG. 2) for a centrifuge having a maximum rotation speed of 4800 RPM. The data presented in this table can be stored in an electronic memory accessible by motor controller 107, such as an on board motor controller memory described above, in order for the motor controller 107 to retrieve a set rotation speed setting corresponding to an operator selected blood collection rate. Although FIG. 3 presents one set of associations, e.g. a linear association, between blood collection rates and rotation speeds, other associations are possible and could easily be computed and stored in the table shown in FIG. 3. For example, empirically collected data points could be represented by an equation satisfying a quadratic fit, an exponential fit, a logarithmic fit, a polynomial fit, a Gaussian model, a logistic model, a power fit, or an exponential association. Moreover, such equations could be stored in a memory of motor controller 107 and executed by its processor to determine corresponding rotation speeds dynamically instead of using a lookup table.

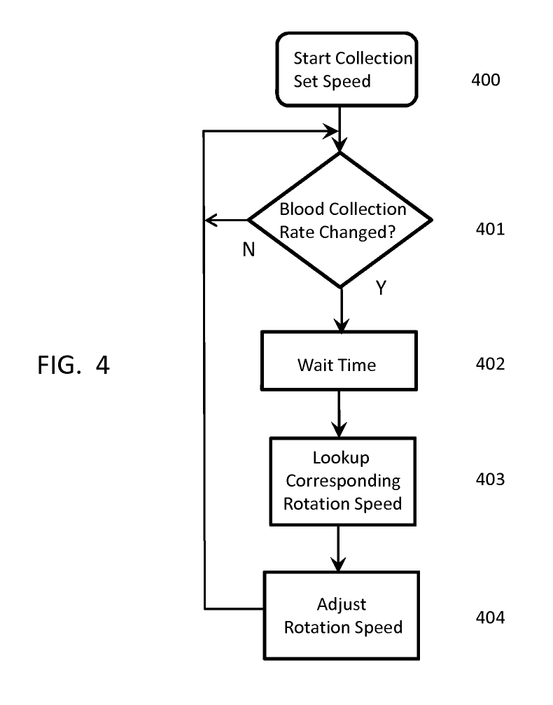

FIG. 4 depicts an exemplary flowchart representing an automatic programmed operation of the motor controller 107. In one embodiment, automatic control over the speed of the motor 101 is managed by motor controller 107, which is initiated at step 400 when blood collection starts at a rate set by an operator of the device. The motor controller detects the setting as input by the operator and automatically sets the rotation speed of the centrifuge 104 after accessing the stored lookup table as shown in FIG. 3. At step 401 motor controller 107 determines whether a blood collection rate set by the operator has been changed. The motor controller 107 is programmed to perform this check every 100 ms. If the blood collection rate has been changed by the operator, the motor controller is preferably programmed to pause for a preset period of time at step 402, typically 60 seconds but it can be programmed to any time period (and is typically not accessible to change by the operator). This wait time is preset because, under normal therapeutic procedure, the operator will make several adjustments to the whole blood collection rate within a short period of time. For example, if a patient undergoing the photopheresis process becomes uncomfortable because the blood collection rate is set too high the operator will lower the blood collection rate. Rather than immediately responding to these temporary adjustments in blood collection rates, motor controller 107 is programmed to wait for a period of time before performing the step of accessing the lookup table to find the rotation speed corresponding to the changed blood collection rate at step 403. At step 404 motor controller 107 adjusts the centrifuge rotation speed according to the corresponding rotation speed found in the lookup table.

It will be apparent to those skilled in the art that various modifications and variations can be made to the methods and processes of this invention. Thus, it is intended that the present invention cover such modifications and variations, provided they come within the scope of the appended claims and their equivalents.

The disclosure of all publications cited above is expressly incorporated herein by reference in their entireties to the same extent as if each were incorporated by reference individually.

### PARTS LIST

100 extracorporeal therapeutic device
101 electric motor
102 outer casing
103 inflow connection
104 inner casing
105 outflow connection
106 internal volume
107 motor controller
400 step for start blood collection and set speed
401 step for determining if blood collection rate is changed
402 step for waiting time
403 step for rotation speed lookup
404 step for adjusting rotation speed

We claim:

1. A method of monitoring operation of an extracorporeal therapeutic device for processing whole blood, the method comprising the steps of:
collecting the whole blood at a preselected collection rate using said extracorporeal therapeutic device;
automatically determining the preselected collection rate of the whole blood in said extracorporeal therapeutic device;
automatically determining a rotation speed of a centrifuge in the extracorporeal therapeutic device, the rotation speed corresponding to the preselected collection rate; and
automatically setting a speed of rotation of the centrifuge in the extracorporeal therapeutic device at the rotation speed corresponding to the preselected collection rate.

2. The method of claim 1, wherein the step of automatically setting the speed of rotation comprises the additional step of automatically increasing a current speed of rotation of the centrifuge in the extracorporeal therapeutic device.

3. The method of claim 1, wherein the step of automatically setting the speed of rotation comprises the additional step of automatically decreasing a current speed of rotation of the centrifuge in the extracorporeal therapeutic device.

4. The method of claim 1, further comprising the additional steps of repeating in sequence the steps of:
automatically determining a current collection rate,
automatically determining the rotation speed corresponding to the current collection rate, and
automatically setting the rotation speed corresponding to the current collection rate.

5. The method of claim 1, wherein the step of automatically determining a rotation speed comprises determining for each of a plurality of collection rates a corresponding rotation speed based on its effectiveness at separating blood components.

6. The method of claim 5, wherein the step of automatically determining a rotation speed further comprises determining a mathematical relationship between the plurality of collection rates and corresponding rotation speeds based on at least one of a quadratic fit, a linear fit, a logarithmic fit, a polynomial fit, a Gaussian model, a logistic model, a power fit, or an exponential association.

7. The method of claim 6, wherein the step of automatically determining a rotation speed further comprises automatically computing the corresponding rotation speed based on a current collection rate and the determined mathematical relationship.

8. The method of claim 5, further comprising the step of storing in a lookup table the determined rotation speeds for each of the plurality of collection rates, including accessing the lookup table during the step of automatically determining the rotation speed.

9. The method of claim 1, wherein the preselected collection rate is set by an operator of the device.

10. An extracorporeal therapeutic system for processing whole blood, the system comprising:
an operator interface for inputting an operator selected collection rate;
an intake for receiving the whole blood at the operator selected collection rate;
a rotating centrifuge for separating components of the whole blood; and
a motor mechanically connected to the centrifuge;
a motor controller for automatically causing the motor to rotate the centrifuge at a predetermined rotation speed corresponding to the operator selected collection rate.

11. The system of claim 10, wherein the motor controller comprises:
a circuit for detecting the selected collection rate and for calculating the predetermined rotation speed based on a mathematical equation; and
memory accessible by the motor controller for storing the equation.

12. The system of claim 10, wherein the motor controller comprises:
a circuit for determining the selected collection rate and for accessing a lookup table to find the predetermined rotation speed; and
memory accessible by the motor controller for storing the lookup table.

13. The system of claim 10, wherein the motor controller comprises a circuit for increasing or decreasing a speed of rotation of the centrifuge.

14. The system of claim 10, further comprising a circuit for periodically checking the operator selected collection rate.

15. The system of claim 11, wherein the equation stored in the memory is at least one of a quadratic fit, an exponential fit, a linear fit, a logarithmic fit, a polynomial fit, a Gaussian model, a logistic model, a power fit, or an exponential association.

16. A blood separation method comprising the steps of:
collecting the blood at a preset collection rate;
spinning the collected blood in a centrifuge at a rotation speed corresponding to the collection rate;
detecting a change in the collection rate; and
adjusting the rotation speed according to the changed collection rate.

17. The method of claim 16, further comprising decreasing or increasing the rotation speed in response to detecting a decrease or increase in the collection rate, respectively.

18. The method of claim 16, further comprising waiting for a preset amount of time between the step of detecting the change and the step of adjusting the rotation speed.

19. The method of claim 16, further comprising generating a tabular listing comprising a plurality of different rotation speeds and a plurality of different collection rates wherein each of the rotation speeds corresponds to one of the collection rates, and wherein the step of adjusting the rotation speed comprises accessing the tabular listing and identifying an adjusted rotation speed corresponding to the changed collection rate.

20. The method of claim 16, wherein the step of adjusting the rotation speed comprises determining the adjusted rotation speed based on a mathematical relationship between the changed collection rate and the adjusted rotation speed.

21. The method of claim 16, wherein the step of detecting a change in the collection rate and the step of adjusting the rotation speed are both performed automatically by a programmed motor controller.
